(19) United States
(12) Patent Application Publication
(10) Pub. No.: US 2006/0210504 A1
(43) Pub. Date: Sep. 21, 2006

(54) Compositions and methods for inhibiting eccrine perspiration in humans

(76) Inventor: Alison B. Lukacsko, West Windsor, NJ (US)

Correspondence Address: MYERS DAWES ANDRAS & SHERMAN, LLP, 19900 MACARTHUR BLVD.,, SUITE 1150, IRVINE, CA, 92612, US

(21) Appl. No.: 11/436,036

(22) Filed: May 16, 2006

Related U.S. Application Data
(62) Division of application No. 10/259,048, filed on Sep. 26, 2002.
(60) Provisional application No. 60/325,105, filed on Sep. 26, 2001.

(51) Int. Cl. A61K 8/28; A61K 8/26; A61K 8/49

(52) U.S. Cl. 424/65; 424/66; 424/68

(57) ABSTRACT

Non-pathological, physiologically normal sweating is inhibited through use of an a muscarinic anticholinergic amine that blocks parasympathetic stimuli from cholinergic nerve fibers to an innerved sweat gland. The anticholinergic agent is included in a form adapted to be topically applied to commonly sweaty areas of the body. Side effects are minimized by employing amines which are charged at physiological pH, thereby minimizing their ability to cross biological membranes. The most prefered anticholinergic agents are salts of quaternary amines (quaternary ammonium salts) which are always charged.

### RELATED APPLICATION

This application relates to and claims priority from U.S. Provisional Application Ser. No. 60/325,105 filed on Sep. 26, 2001 entitled “COMPOSITIONS AND METHODS FOR INHIBITING ECCRINE PERSPIRATION IN HUMANS,” the disclosure of which is incorporated herein by reference.

### BACKGROUND OF THE INVENTION

1. Field of the Invention

The invention relates generally to antiperspirant compositions and uses thereof.

2. Description of Prior Art and Related Information

Currently available metal salt antiperspirants work by inhibiting the expression of eccrine sweat through the sweat duct onto the surface of the skin. The prevailing theory is that this antiperspirant action represents physical blockage caused by the formation of a plug composed of insoluble metal salts which precipitate within the duct. Generally, the metal salt antiperspirant is applied to the surface of the skin, migrates into the sweat duct where pH of the sweat renders the salt insoluble so that it fills the duct. Eventually, dilution of the plug from expressed sweat washes it out and sweat expression onto the skin is restored. Other theories of effectiveness include protein coagulation, irritation and local swelling, and reaction of the metal salt with skin keratin to form fibril plugs. All of these processes would be expected to result in inflammation or some other response by the affected tissues, the net result of which is blockage to sweat flow.

Therefore, it is not surprising that use of such metal salt antiperspirants can often. lead to skin irritation. The presently available antiperspirants may also leave a whitening on the skin that is generally unsightly and sticky. Even worse, such antiperspirants often deposit in fabric leaving stains on the fabric that may be difficult or impossible to remove. Delicate fabrics may be weakened as well.

Further, the metal salt antiperspirants may not be fully effective. At best, commercial products, depending on form and active ingredient, range from 20-50% inhibition. Efficacy would be further limited—especially if the user reduces the amount or frequency of application to reduce irritation and/or fabric damage. This results in embarrassing wet spots on clothes and even malodor. Further the sweat may stain or damage fabrics leading to a “catch 22” situation where clothing is ruined in either instance.

The need for a truly effective antiperspirant that is aesthetically pleasing, non-irritating to skin and non-damaging to clothing is palpable.

### SUMMARY OF THE INVENTION

In accordance with the present invention, structures and associated methods are disclosed which address these needs.

In one aspect, an antiperspiration composition is provided for topical application to control sweating. The composition comprises an anticholinergic amine in a weight per volume between 1% and 5% of the composition, and a vehicle. The vehicle comprises an anhydrous solution, a suspension of the amine in a hydrophobic matrix, water, or an alcohol. The composition may further comprise a fragrance, an anti-microbial agent, a skin penetration enhancer, and/or an emulsifier. The anticholinergic amine preferably has a pKa greater than 9.0. The anticholinergic amine is charged at a physiologic pH.

In preferred embodiments, the anticholinergic amine has a weight per volume between 2% and 3% of the composition. The anticholinergic amine may comprise a quaternary amine, preferably glycopyrrolate. Other anticholinergic quaternary amines may also be employed, such as methscopolamine, homatropine, methantheline, propantheline, ambutonium, benzilonium, dibutoline, diphemanil, emepronium, blycopyrronium, isopropamide, lachesine, mepenzolate, methantheline, oxyphenonium, propantheline, ipatropium, n-methyl atropine, and n-methylhyoscine methobromide.

In another aspect, an antiperspiration composition adapted for topical application to control sweating comprises an anticholinergic quaternary amine in a weight per volume between 2% and 3% of the composition, and a vehicle. The vehicle may comprise an anhydrous solution, a suspension of the amine in a hydrophobic matrix, water, or an alcohol.

The composition further comprises a fragrance, an anti-microbial agent, a skin penetration enhancer, and/or an emulsifier. The anticholinergic quaternary amine may comprise glycopyrrolate, or methscopolamine, homatropine, methantheline, propantheline, ambutonium, benzilonium, dibutoline, diphemanil, emepronium, blycopyrronium, isopropamide, lachesine, mepenzolate, methantheline, oxyphenonium, propantheline, ipatropium, n-methyl atropine, and n-methylhyoscine methobromide.

In a further aspect, an antiperspiration composition adapted for topical application to control sweating comprises a vehicle, and an anticholinergic amine in combination with a metal salt antiperspirant. The metal salt antiperspirant may comprise aluminum, zirconium, a mixture thereof, and more.

A method is provided for inhibiting non-pathological sweating. The method comprises the steps of topically applying an anticholinergic composition to a body area, penetrating a skin of the body area with the anticholinergic composition, and blocking the result of sympathetic cholinergic nerve fiber releasing acetylcholine to an innerved sweat gland with the anticholinergic composition.

The step of topically applying an anticholinergic composition to a body area comprises the step of topically applying to the body area an anticholinergic quaternary amine having a weight per volume between 1% to 5%. The step of topically applying an anticholinergic quaternary amine to the body area comprises the step of topically applying glycopyrrolate to the body area.

The method further comprises the steps of ensuring that the anticholinergic composition is charged at a physiological pH to prevent the anticholinergic composition from being absorbed systemically, delaying the penetration step until local perspiration occurs, and/or eliminating sweat odors with an anti-microbial agent.

The method may also comprise the step of keeping pores of the penetrated skin free of plugs. Alternatively, where a preferred anticholinergic composition is combined with a metal salt antiperspirant, the method may comprise the step of plugging the pores of the penetrated skin.

In summary, non-pathological, physiologically normal sweating is inhibited through use of an a muscarinic anticholinergic amine that blocks parasympathetic stimuli from cholinergic nerve fibers to an innerved sweat gland. The anticholinergic agent is included in a form adapted to be topically applied to commonly sweaty areas of the body. Side effects are minimized by employing amines which are charged at physiological pH, thereby minimizing their ability to cross biological membranes. The most preferred anticholinergic agents are salts of quaternary amines (quaternary ammonium salts) which are always charged.

### DETAILED DESCRIPTION OF THE PREFERRED EMBODIMENTS AND BEST MODE OF INVENTION

Methods and compositions are provided herein for inhibiting normal, everyday sweat in humans. Such eccrine sweat typically occurs in areas such as the armpits, feet, back, face, neck, groin and other more sweaty parts of the body.

A preferred embodiment comprises topically applying to such sweaty parts of the body a composition comprising an anticholinergic agent that interrupts the conduction of the homeostatic nerve signal from the brain to the sweat gland—the very signal that stimulates the gland to secrete sweat. Since the eccrine sweat glands are innerved by sympathetic cholinergic nerve fibers which release acetylcholine resulting in the production of sweat, the message to sweat can, therefore, be interrupted by anticholinergic agents, thereby producing anti-perspirancy.

In the preferred embodiments, delivery of the sweat blocker to the secretory portion of the sweat gland is accomplished with minimal systemic exposure so as to avoid undesirable side effects. Since anticholinergic drugs are generally very potent drugs, small doses within the systemic circulation can produce undesirable side effects, such as dry mouth, decreased bronchial secretion, and more serious complications such as increased papillary diameter (i.e., dilation of the pupils of the eye), decreased urination, increased heart rate and CNS depression. In the preferred embodiments, however, control of sweat is accomplished without the systemic side effects characteristic of anticholinergic drugs. Topical administration at the site at which the drug effect is desired (the armpit, palm of hand, sole of foot) optimizes intradermal delivery to the eccrine gland within the dermis. Any systemic exposure to the drug is diluted by the whole body volume so as to avoid systemic side effects. The side effect profile may, further, be minimized by selection of anticholinergic compounds with differing physical-chemical properties. Some compounds, for example, are charged at physiologic pH, minimizing their ability to cross biological membranes, thereby making it unlikely that topical administration will result in uptake by the circulatory system resulting in systemic effects. As is well understood by those of skill in the art the charge of a compound at a given pH can be determined from the compound's pKa. Quaternary ammonium salts are always charged in solution at physiological pH.

The product formulation would be dependent upon what is appropriate or desired for the form. It may be opaque, translucent or clear. It also may be anhydrous or water based, utilizing such combination of components as provides the desired profile of dose and aesthetics. In general, it will include a vehicle/carrier, dispersant, emollient, fragrance, surfactant and structurants.

The formulation will likely include a base such as stearic acid, water, wax and/or silicone fluid; and at least one anticholinergic drug at concentrations ranging from 0.0001% to 20% w/w, with a preferred range of 0.001% to 10% and a more preferred range of 0.01% to 5%. The active ingredient may be a free base, salt or analogue of the drug. The term glycopyrrolate as used herein is intended to be broader than the compound of that name unless indicated otherwise; it is a quaternary ammonium compound that also includes analogues capable of inhibiting cholinergically mediated sweat secretion wherein the chemical structure has been modified to introduce, modify or remove or change functionalities of the structure. For example, such modification can result in the removal of an OH functional group and the like. Insofar as the modified molecule can inhibit perspiration, it is hereby encompassed. By virtue of the presence of a quaternary amino group, compounds of this invention readily form salts. The drug is acceptable with a counter salt.

Acceptable countersalts of quaternary amines can be prepared from inorganic and organic acids. These may include hydrochloric, hydrobromic, sulfuric, nitric, phosphoric, glycolic, pyruviic, oxalic, malic, malonic, succinic, maleic, fumaric, tartaric, citric, benzoic, cinnamic, mandelic, methanesulfonic, ethanesulfonic, p-toluene-sulfonic, salicylic acids as well as hydrogen fluoride, hydrogen iodide, and the like. The counter ion chosen can be important for producing a solution that is near to a neutral pH.

The formulation may comprise materials which functionally serve as structurants and/or structure enhancers or modifiers, emollients, surfactants, co-surfactants, fragrances, emulsifiers, dispersants, suspending agents, wash-off agents, controlled release agents, penetration enhancing, controlling or release agents. Materials within a given functional group may be combined to balance the benefits. For example, low and high melt point waxes may be combined to optimize manufacturability. Likewise, polyethylene waxes may be used in combination with classic organic materials. Similarly, silicone and silicone derivatives may be combined to control and vary product properties. It may include materials with activities such as keratolytic, antioxidant, anti-inflammatory, deodorant, anti-fungal, anti-bacterial, moisturization, barrier protection, UV-protection, depilation, skin conditioning, etc. or other activities which are intended to impart benefit to the skin itself or modify other functions of the sweat gland/sweat duct unit. It may also be used in combination with metal salt antiperspirants to enhance their efficacy via a different mechanism of action.

The drug may be incorporated into reservoir or other type of “patch” or plaster or embedded in a matrix for controlled release e.g. sole insert to deliver to foot or sock or glove to deliver to sole/palm. Such systems have an added advantage of focusing delivery to the desired body surface while protecting the outer surface from unintentional or undesirable exposure. The drug may be delivered in a film forming matrix in which case the film layer develops once the product is applied. Such film may create occlusion to enhance penetration or may simply create a “reservoir” of drug on the skin surface to drive movement through the epidermis into the dermis wherein it acts on the sweat gland.

Numerous anticholinergic materials have been identified and are in clinical use or development. In general, these can be categorized as primary, secondary, tertiary or quaternary amines. Those that are preferred for the envisioned application must penetrate the skin without irritation. It is an advantage if they are charged within the body so as not to easily cross biological membranes. Glycopyrollate (sensu stricto) is an example of such a material. It is a quaternary amine which carries a positive charge at physiological pH. Glycopyrrolate may be utilized in a simple solution or in an emulsion, dispersion, suspension, liquid crystalline, cream, gel or ointment base. It may be used neat or encapsulated partially or in part (e.g., a clathrate) or in combination e.g., to control release rate or its dose time profile. Given its charged state and the related resistance to crossing biological membranes, it is unexpected that it would be effective via the transdermal route of administration.

Testing

The antiperspirant efficacy and safety of glycopyrrolate, a quaternary ammonium salt, was tested pursuant to protocol prescribed by the Food and Drug Administration, 21CFR Part 10.90 The OTC Antiperspirant Tentative Final Monograph. So as to separate vehicle from drug effects, glycopyrrolate was presented as a simple solution in varying concentrations of 0.3%, 1% and 3% weight per volume. In each treatment group as listed below, the glycopyrrolate solution and a placebo of distilled water were applied to the axilla of healthy female volunteers. In particular, the placebo was applied to one armpit while the test solution was applied to the other armpit of a given volunteer. The designation of armpits (namely, right or left) for the application of placebo vs. solution was randomized according to the FDA protocol. In the placebo Group B, distilled water was applied to both axilla of each volunteer. Treatments were applied once daily for five consecutive days.

The groups comprised the following:
Group A—0.3% glycopyrrolate versus distilled water;
Group B—Placebo (distilled water applied to both axilla);
Group C—1% glycopyrrolate versus distilled water;
Group D—3% glycopyrrolate versus distilled water;

The clinical study was conducted among 37 healthy female volunteers. The testing further conformed to ICH guidelines and the requirements of the 1964 Declaration of Helsinki and its subsequent amendments. The study was a pilot, single-center, randomized, controlled, double-blind assessment of the safety, tolerability and antiperspirant efficacy of glycopyrrolate when applied to the axilla of healthy female volunteers.

The study was conducted in two cohorts of volunteers. The first cohort comprised fourteen (14) subjects, with five (5) subjects assigned to placebo Group B and ten (10) subjects assigned to 0.3% Group A.

Nine (9) days following the completion of the first cohort, a second cohort of twenty-three (23) subjects were tested, with three (3) subjects assigned to placebo Group B, ten (10) subjects assigned to 1% Group C, and ten (10) subjects assigned to 3% Group D.

The antiperspirant efficacy evaluation was performed approximately one hour after the fifth treatment using a standard hot room protocol. Safety and tolerance were measured by assessment of blood chemistry and hematology, electrocardiograms, daily monitoring of heart rate, blood pressure and body temperature, assessment of visual effects and dermal irritancy, and subjective assessment of tolerance. No serious adverse events occurred during the study. All adverse events during the study were reviewed and determined by the principal investigator and the attendant cardiologist to be clinically non-significant. There were no signs of pupillary dilation or other systemic symptoms of anticholinergic agents.

Placebo Group B and 0.3% Group A showed no statistically significant reduction in sweat production while 1% Group C and 3% Group D showed significant sweat reduction. In particular, 1% Group C showed mean reduction of 32.85%±9.81 while 3% Group D showed reduction of 54.72%±20.23. Both the 1% and 3% groups met the binomial criteria of 95% confidence with at least 50% of the population showing at least 20% reduction in sweating. The range of effect in the 1 and 3% treatment groups was −16-to 54 and −13 to 90% inhibition, respectively. This degree of efficacy far exceeds that which can be achieved with conventional commercial products which generally range from 20-50% mean percent inhibition.

Under the conditions of the study, it is concluded that glycopyrrolate in simple solution shows a dose-dependent antiperspirant effect with the highest efficacy observed at the 3% concentration. Low concentrations such as 0.3% were essentially ineffective, although even there two subjects enjoyed 0.50% inhibition.

Anticholinergic Quaternary Amines (ACQA)

Although a variety of anticholinergic compounds are useable in the present invention, ACQAs, such as glycopyrrolate, are inherently water soluble, and, thus, may be either suspended in a hydrophobic milieu such as a silicone or wax based matrix, perhaps via emulsification, or dissolved in a hydrophilic or aqueous milieu either as a solution or gel. Preferred end products may come in a variety of forms and matrices, including sticks, roll-ons, creams, pumps, aerosols, gels, powders and soft solids. The topical application provided by such forms would preferably occur in an open system, namely, where patches are omitted and the ACQA is active while the treated area is exposed to temperatures, light and air.

The eccrine sweat glands are the preferred target for the prevention of sweating. The preferred method of topically applying ACQAs, therefore, requires that a sufficient amount of ACQA penetrate through the skin to reach the sweat gland which lies in the dermis. This treatment provides an intradermally delivered antiperspirant. Though not necessary to the preferred embodiments, transport of the ACQA may be enhanced by skin penetration enhancers, such as water miscible organic solvents.

If ACQAs are not used, amines having pKa's higher than about 9.0 or 9.5 are preferred to ensure that substantially all of the molecules are ionized at normal physiological pH. Molecules that are charged at physiological pH, are essentially unable to pass cell membranes so that topical administration is unlikely to result in systemic effects. It will be appreciated that an ACQA, such as glycopyrrolate, having properties for being inherently poorly absorbed (i.e., hydrophilic and charged at physiological pH) is unexpectedly-effective at accomplishing antiperspirancy. In other words, it is unexpected that an inherently poorly absorbed molecule (i.e., one unable to pass through cell membranes) would be able to penetrate through the skin and into sweat glands. While not wishing to be held to any particular explanation, the inventor suspects that the route of access of the ACQAs is through the sweat duct itself, namely that the molecules dissolve in the sweat within the ducts and diffuse into the innervated region of the gland where they block cholinergic stimulation.

Since ACQAs are hydrophilic, the onset of local perspirancy would not void activity. Conversely, an ACQA, such as glycopyrrolate, may be provided in a certain hydrophobic form or matrix to provide a desirable, delayed response. Such a timed-release response would occur when local perspirancy first diffuses into the hydrophobic matrix (present as a film on the skin surface), which causes the ACQAs to be dissolved, whereby they are able to diffuse to and suppress the activity of the sweat gland.

Though the examples below comprise glycopyrrolate, it is to be expressly understood that a plurality of ACQAs may be employed, including, but not limited to, the following: methscopolamine, homatropine, methantheline, propantheline, ambutonium, benzilonium, dibutoline, diphemanil, emepronium, blycopyrronium, isopropamide, lachesine, mepenzolate, methantheline, oxyphenonium, propantheline, ipatropium, n-methly atropine, n-methylhyoscine methobromide, and similar anticholinergic amines. In fact, glycopyrrolate may be substituted in the examples below with any single ACQA or combinations of ACQAs having properties for controlling eccrine sweat at concentrations which do not cause serious side effects. The required concentrations may vary given the varying but consistently high potency of these materials in general. If agents that are not ACQAs are employed, it is important to select a compound with a pKa indicating that the compound will be substantially entirely charged at physiological pH.

Additional Materials

It is to be expressly understood that the preferred embodiments of the antiperspirant composition may include additional materials and components including vehicles/carriers and mixtures thereof, dispersants, emollients, skin penetration enhancers, fragrances, anti-microbial agents, odor absorbers, odor neutralizers, surfactants, structurants, emulsifiers, sensory modifiers, coloring agents, UV protectants and more. U.S. Patent Application Publication No. 2002/0037264 entitled “ANTIPERSPIRANT AND DEODORANT PRODUCTS AND METHODS FOR THEIR USE” (the “'264 application”) is incorporated by reference as if fully set forth herein.

It will be appreciated that the preferred embodiments of the antiperspirant compositions may also be incorporated with a metal salt antiperspirant to provide a synergistic combination. As examples and not by way of limitations, metal salt antiperspiration compositions may include those disclosed in the '264 application, such as aluminum, zirconium, mixed aluminum/zirconium salts, and more. Antiperspirancy is thus accomplished by both interruption of the conduction of the homeostatic nerve signal as well as blocking of skin pores. In such cases, a lower concentration of the preferred anticholinergic compositions may be employed.

### EXAMPLES

The following compositions are provided as examples and not by way of limitations. Percentages are indicated as weight per volume.

### Example 1

### Anhydrous Clear Solid

| Ingredient | % |
|---|---|
| Glycopyrrolate | 2 |
| Propylene glycol | 74.5 |
| PPG-3 isosteareth-9 | 8 |
| Propylene carbonate | 6 |
| Dipropylene glycol | 4 |
| Dibenzylidene sorbitol | 3 |
| Dimethicone copolyol | 1.5 |
| Fragrance | 1 |

### Example 2

### Anydrous Solid

| Ingredient | % |
|---|---|
| Glycopyrrolate | 2 |
| Cyclomethicone | 50.5 |
| Diisopropyl sebacate | 20.7 |
| Stearyl alcohol | 11.4 |
| Dimethicone | 3 |
| Castor wax | 2.9 |
| Silica | 0.5 |
| Polyethylene | 1 |
| Aluminum silicate | 4 |
| Fragrance | 1 |
| Alkyl benzoate | 3 |

### Example 3

### Anydrous Suspension Cream

| Ingredient | % |
|---|---|
| Glycopyrrolate | 2 |
| Cyclomethicone, D5 | 41 |
| Permethyl 103A | 20 |
| Propylene Carbonate | 2 |
| Quaternium hectorite | 8 |
| Talc | 10 |
| Polyethylene | 16 |
| Fragrance | 1 |

### Example 4

### Clear Water in Oil Emulsion Gel/Roll-On

| Ingredient | % |
|---|---|
| Glycopyrrolate | 2 |
| Water | 45 |
| Propylene glycol | 21.5 |
| Ethanol | 11 |
| Volatile dimethicone | 7 |
| Dimethicone, 1000 cs | 3 |
| Dimethicone copolyol | 1 |
| Cyclomethicone | 9 |
| Fragrance | 0.5 |

### Example 5

### Clear Solid

| Ingredient | % |
|---|---|
| Glycopyrrolate | 2 |
| Propylene glycol | 50 |
| Water | 34.3 |
| Sodium stearate | 8 |
| Procetyl AWS | 3 |
| Sodium chloride | 0.2 |
| Dimethicone copolyol | 1.5 |
| Fragrance | 1 |

### Example 6

### Anydrous Nonresidue Solid

| Ingredient | % |
|---|---|
| Glycopyrrolate | 2 |
| N-lauroyl-L-glutamic acid-di-n-butylamide | 41 |
| 12-hyrdoxystearic acid | 7 |
| Cyclomethicone, D5 | 42 |
| Polyisobutene | 15 |
| Isopropyl myristate | 20 |
| Polyethylene | 8 |
| Fragrance | 1 |

### Example 7

### Anydrous Soft Solid/Cream

| Ingredient | % |
|---|---|
| Glycopyrrolate | 2 |
| Cyclomethicone | 64.7 |
| Glyceryl tribehenate | 5 |
| C18-36 triglycerides | 1.3 |
| Talc | 10 |
| Polyethylene | 16 |
| Fragrance | 1 |

### Example 8

### Oil in Water Emulsion Cream/Lotion

| Ingredient | % |
|---|---|
| Glycopyrolate | 2 |
| Cyclomethicone, D5 | 4.5 |
| Dimethicone copolyol | 0.5 |
| Isocetyl stearate | 3 |
| Cetearyl alcohol/Ceteareth 20 | 2.5 |
| Myristyl Myristate | 2.5 |
| Water | 83.7 |
| Carbomer 934 | 0.3 |
| Triethanolamine | 0.5 |
| Fragrance | 0.5 |

It will be appreciated that the preferred antiperspirant methods and compositions provide several advantages over conventional antiperspirants. A pleasant aesthetic touch is provided as the preferred embodiments do not leave a sticky, tacky, oily or greasy feeling. It will further be appreciated that the preferred embodiments do not irritate the skin. No wax residues result from using the preferred embodiments. Instead, the preferred embodiments go on to the target body area clearly without whitening the skin or clothing.

Superior efficacy is accomplished as the desired antiperspirancy does not rely on physical plugs filling in pores of the skin, although a synergistic effect may be accomplished with the plugging of pores, as discussed above. The preferred embodiments are configured to remain stable at high temperatures. Duration of antiperspirancy is increased.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what incorporates the essential idea of the invention.

1-37. (canceled)

38. An antiperspirant composition adapted for topical application to control sweating, comprising:
a hydrophilic anticholinergic quaternary amine at a weight per volume of between about 1% and about 5% of the composition; and
a vehicle.

39. The composition of claim 38, wherein the hydrophilic anticholinergic quaternary amine has a pKa greater than 9.0.

40. The composition of claim 38, wherein the hydrophilic anticholinergic quaternary amine is present at a weight per volume between about 2% and about 3% of the composition.

41. The composition of claim 38, wherein the vehicle comprises a material selected from the group consisting of water, an alcohol, an anhydrous solution and a hydrophobic matrix with said quaternary amine suspended therein.

42. The composition of claim 38, further comprising an additive selected from the group consisting of a fragrance, an anti-microbial agent, an emulsifier and a skin penetration enhancer.

43. The composition of claim 38, wherein the hydrophilic anticholinergic quaternary amine comprises glycopyrrolate.

44. The composition of claim 38, wherein the hydrophilic anticholinergic quaternary amine is selected from a group consisting of ambutonium, benzilonium, dibutoline, diphemanil, emepronium, isopropamide, mepenzolate, methantheline, oxyphenonium and propantheline.

45. An antiperspirant composition adapted for topical application to control sweating, comprising:
a hydrophilic anticholinergic quaternary amine;
a metal salt antiperspirant; and
a vehicle.

46. The composition of claim 45, wherein the metal salt antiperspirant comprises aluminum.

47. The composition of claim 45, wherein the metal salt antiperspirant comprises zirconium.

48. The composition of claim 45, wherein the hydrophilic anticholinergic quaternary amine is present at a weight per volume between about 1% and about 3% of the composition.

49. The composition of claim 45, wherein the vehicle comprises a material selected from the group consisting of water, an alcohol, an anhydrous solution and a hydrophobic matrix with said amine suspended therein.

50. The composition of claim 45, further comprising an additive selected from the group consisting of a fragrance, an anti-microbial agent, an emulsifier and a skin penetration enhancer.

51. The composition of claim 45, wherein the hydrophilic anticholinergic quaternary amine comprises glycopyrrolate.

52. The composition of claim 45, wherein the hydrophilic anticholinergic quaternary amine is selected from a group consisting of ambutonium, benzilonium, dibutoline, diphemanil, emepronium, isopropamide, mepenzolate, methantheline, oxyphenonium and propantheline.

53. An antiperspirant composition adapted for topical application to control sweating, comprising:
glycopyrrolate at a weight per volume of between about 1% and about 5% of the composition; and
a vehicle.

54. The composition of claim 53, wherein the glycopyrrolate is present at a weight per volume between about 2% and about 3% of the composition.

55. The composition of claim 53, wherein the vehicle comprises a material selected from the group consisting of water, an alcohol, an anhydrous solution and a hydrophobic matrix with the glycopyrrolate suspended therein.

56. The composition of claim 53, further comprising an additive selected from the group consisting of a fragrance, an anti-microbial agent, an emulsifier and a skin penetration enhancer.

57. The composition of claim 53 further comprising a metal salt antiperspirant.
